(19) United States
(12) Patent Application Publication
(10) Pub. No.: US 2025/0034645 A1
(43) Pub. Date: Jan. 30, 2025

(54) SYSTEMS AND METHODS FOR INFERRING GENOTYPES OF BIOLOGICAL SAMPLES

(71) Applicant: Michael CHRISTENSEN, Brandenberg (DE)

(72) Inventor: Michael CHRISTENSEN, Brandenberg (DE)

(21) Appl. No.: 18/782,686

(22) Filed: Jul. 24, 2024

Related U.S. Application Data
(60) Provisional application No. 63/515,305, filed on Jul. 24, 2023.
(60) Provisional application No. 63/674,912, filed on Jul. 24, 2024.

(51) Int. Cl. C12Q 1/6883; G16B 40/20; G16H 50/20

(52) U.S. Cl. CPC C12Q 1/6883; G16B 40/20; G16H 50/20

(57) ABSTRACT

Disclosed herein are methods, systems, and devices for inferring genetic information, or genotypes, of a subject based on low-coverage, or otherwise incomplete, genotype data from the individual and known genetic information of the subject's parents. In particular, disclosed herein are methods for generating a predicted, comprehensive genome of an offspring regardless of the quality of or gaps in coverage in the individual's data and/or the parental data.

### RELATED APPLICATIONS

This application claims the benefit of priority to U.S. Provisional Application No. 63/674,912 filed Jul. 24, 2024 and U.S. Provisional Application No. 63/515,305 filed Jul. 24, 2023, the entireties of which are incorporated herein by reference.

### FIELD OF THE DISCLOSURE

Various embodiments of the present disclosure relate generally to systems and methods for inferring genotypes of offspring from incomplete genetic data and estimated parental haplotypes. More specifically, the present disclosure utilizes novel systems and methods to infer offspring genotype at any genomic position for which the offspring genotype data is missing or incomplete.

### BACKGROUND

Various genotyping technologies and techniques may be used to infer the genome of an organism, including array genotyping and sequencing. These technologies differ in their methodologies, applications, and the type of genetic information they provide. Whole genome sequencing may offer comprehensive coverage across the entire genome, capable of detecting both known and novel genetic variants. However, it may be more resource-intensive in terms of cost and data processing. Array genotyping, in contrast, may be highly efficient at detecting specific, predetermined genetic variants across many samples, but may be limited to analyzing only those variants included on the array. While both technologies have proven valuable in genomic research and clinical applications, they each may have limitations. Sequencing, particularly at lower depths of coverage, may introduce uncertainties in variant calling. Array genotyping, despite its efficiency, may not detect variants outside its predetermined set and may miss rare or structural variants. Furthermore, both technologies may face challenges when working with minimal biological samples, such as in preimplantation genetic testing, where the amount of genetic material may be extremely limited. These limitations may create a need for advanced computational methods that can enhance and extend the capabilities of existing genotyping technologies, particularly in scenarios where genetic material may be scarce or when comprehensive genomic information may be crucial for decision-making. The present disclosure addresses these and other needs in the art.

Utilizing machine learning models and known parent genetic information may allow for the generation of comprehensive genomic information from a biological sample from one or more offspring. However, until now, it has been considered impossible to accurately infer the complete genome of one or more offspring from ultra-low coverage sequencing data such as generated in preimplantation genetic testing. The systems and methods disclosed herein may use machine learning models, including statistical models, and known parental genetic information to generate comprehensive genetic information on one or more offspring while accounting for uncertainty. Advantageously, the disclosed systems and methods are compatible with various types of genotyping data, including array genotyping data and sequencing data, including low and ultra-low coverage sequencing data, and can produce comprehensive genomic information with enhanced accuracy over known existing techniques.

### SUMMARY

Provided herein are methods for generating a predicted genome of a subject. In some aspects, the method may comprise: receiving a biological sample from the subject, wherein the subject is an offspring of a first parent and a second parent; genotyping the biological sample to produce offspring genotype data; providing, to a machine learning model, the offspring genotype data, a first parental genotype data from the first parent, and a second parental genotype data from the second parent to determine a probability distribution; and receiving, from the machine learning model, a predicted genome of the subject based on the probability distribution. In some embodiments, the method may further comprise receiving, from the machine learning model, at least one predicted polygenic risk score for the subject based on an expected genotype at each base position in the predicted genome and/or based on sampled inheritance vectors. In some aspects, the at least one predicted polygenic risk score for the subject is determined by: using the probability distribution to determine an expected genotype of each offspring at each position in a genome for one or more offspring of the first parent and the second parent, and/or sampling inheritance vectors in proportion to their probability based on estimated parental haplotypes and observed genotype data from the one or more offspring; generating one or more offspring polygenic risk scores based on the expected genotype at each position in a genome for one or more offspring and/or based on the sampled inheritance vectors; and determining the at least one predicted polygenic risk score for the subject based on the one or more offspring polygenic risk scores.

Further provided herein are methods for generating a predicted genome of a subject. In some aspects, the method may comprise: receiving an offspring genotype data of the subject, a first parental genotype data of a first parent, and a second parental genotype data, wherein the subject is an offspring of the first parent and the second parent; determining, using a machine learning model, a probability distribution for an offspring genotype based on the offspring genotype data, the first parental genotype data, and the second parental genotype data; generating, using the machine learning model, a predicted offspring genome based on the probability distribution; and outputting the predicted offspring genome. In some embodiments, the method may comprise outputting at least one predicted polygenic risk score for the subject. In some aspects, outputting the at least one predicted polygenic risk score of the subject may comprise: using the probability distribution to determine an expected genotype of each offspring at each position in a genome for one or more offspring of the first parent and the second parent, and/or sampling inheritance vectors in proportion to their probability based on estimated parental haplotypes and observed genotype data from the one or more offspring; generating one or more offspring polygenic risk scores based on the expected genotype at each position in a genome for one or more offspring and/or based on the sampled inheritance vectors; determining the at least one predicted polygenic risk score for the subject based on the one or more offspring polygenic risk scores; and outputting the at least one predicted polygenic risk score.

In some embodiments, the first parental genotype data may comprise a complete genome of the first parent, the second parental genotype data may comprise a complete genome of the second parent, and the offspring genotype data may comprise a partial genome of the subject. In some embodiments, the offspring genotype data may be produced by array genotyping and/or sequencing of a biological sample from the subject.

In some embodiments, the machine learning model may comprise a Hidden Markov Model. In some embodiments, the offspring genotype data may comprise an average coverage of less than one read per base position. In some embodiments, the parental genotype data may comprise information at one or more additional base positions than the offspring genotype data.

In some embodiments, the method may comprise analyzing the predicted offspring genome to determine a probability that the subject has or will develop one or more genetic disorders. In some aspects, the one or more genetic disorders may arise from chromosome microdeletions, chromosome aneuploidies, single gene conditions, or other genetic variations. In some aspects, the one or more genetic disorders may include Angelman Syndrome, DiGeoge/VCF, Prader-Willi Syndrome, Williams Syndrome, Down Syndrome, Klinefelter Syndrome, Trisomy 18, Trisomy 13, Turner Syndrome, Ehlers-Danlos Syndrome, Fragile X Syndrome, Marfan Syndrome, Neurofibromatosis Type 1, Noonan Syndrome, Osteogenesis Imperfecta, Phenylketonuria, Rett Syndrome, Smith-Lemli-Opitz Syndrome, Tuberous Sclerosis, and Russell-Silver Syndrome.

Further provided herein are systems, computer-implemented systems, and devices for implementing or performing the methods described herein.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a block diagram of a system for inferring genotypes from biological samples, according to techniques discussed herein.

FIG. 2 is a block diagram depicting an exemplary method for receiving a predicted genome of an offspring, according to techniques discussed herein.

FIG. 3 is a block diagram depicting an exemplary method for generating a predicted genome of an offspring, according to techniques discussed herein.

FIG. 4 is a block diagram depicting an exemplary method of operation of a machine learning model for generating a predicted genome of an offspring, according to techniques discussed herein.

FIG. 5 is a block diagram depicting an exemplary device for generating a predicted genome of an offspring, according to aspects discussed herein.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

### Definitions

As used herein, the term “biological sample” or “sample” refers to one or more cells or tissue or fluids obtained from a subject. A biological sample may comprise nucleic acid molecules, such as deoxyribonucleic acid (DNA) molecules, made up of nucleic acid bases. For example, DNA molecules are made up of nucleic acid bases adenine (A), guanine (G), cytosine (C), and thymine (T). As used herein, the term “minimal biological sample” refers to a biological sample comprising a very low number of cells. For example, a minimal biological sample may contain no more than twenty cells, no more than ten cells, no more than five cells, no more than four cells, no more than three cells, no more than two cells, or no more than one cell.

As used herein, unless stated otherwise, the terms “offspring” or “progeny” refer to an organism that is produced by the union of gametes (sperm and egg cells) from biological parents, including embryos that have not yet been implanted.

As used herein, unless specified otherwise, the term “sequence” refers to the nucleic acid sequence of a nucleic acid molecule (i.e., oligonucleotide, polynucleotide). For example, the nucleic acid sequence of a DNA molecule may be referred to as a DNA sequence.

As used herein, the terms “gene” or “gene sequence” refer to a DNA sequence encoding an amino acid sequence of a particular polypeptide (i.e., protein). For example, a particular protein may be built from a sequence of amino acids encoded by a DNA sequence, and that DNA sequence may be referred to as the gene encoding that particular protein.

As used herein, the term “genomic position” or “locus” refers to a position within a genome (e.g., on a particular chromosome). A genomic position may refer to a single nucleotide position or a group of nucleotide positions on a particular chromosome within a genome. Alternatively, a genomic position may refer to one or more genomic coordinates and/or a span of genomic coordinates (e.g., within a reference sequence or genome). Diploid cells typically have two copies (i.e., two alleles) of a gene or nucleotide at each genomic position within its genome, while haploid cells typically have only one copy (i.e., one allele) at each position.

As used herein, unless stated otherwise, the term “chromosome” refers to a structure found in the nucleus of a eukaryotic cell, made up of DNA molecules bound on histone proteins. Typically, in humans, each cell nucleus contains 23 pairs of chromosomes, and each pair of chromosomes contains one chromosome inherited from a male parent and one chromosome inherited from a female parent. Some cells may exhibit chromosomal abnormalities resulting in missing or extraneous chromosomes.

As used herein, the term “haplotype” refers to a single copy of the chromosome carried by an individual diploid organism, with each diploid organism having inherited one haplotype from each parent. A haplotype comprises a DNA sequence that may be mapped to a reference genome (e.g., a human reference genome) with coordinates given by the number of bases from the start of chromosome to the end. Haplotypes may be estimated from genotyping technologies such as genotyping arrays and genome sequencing technologies that produce reads, as described herein.

As used herein, the term “recombination” refers to a phenomenon occurring during meiosis, in which different parental haplotypes “cross-over” and exchange genetic material to produce haploid gametes (e.g., sperm cells or egg cells). As a result, an offspring haplotype inherited from a parent (e.g., mother or father) may be comprised of one grandparent's haplotype in some sections and the other grandparent's haplotype in other sections (the maternal grandparents for the maternally-inherited haplotype, the parent grandparents for the paternally-inherited haplotype), a characteristic that may be referred to as “mosaic.”

As used herein, the terms “preimplantation genetic testing for aneuploidy”, “PGT-A”, “preimplantation genetic screening” refer to screening tests for evaluating genetic material (e.g., chromosomes, DNA, etc.) in developing pre-natal offspring cell cluster. Specifically, PGT-A uses genome sequencing data—typically ultra-low coverage—to screen for the presence of any missing or extraneous chromosome material. PGT-A may be performed in the context of in vitro fertilization and/or other assisted reproduction technologies.

As used herein, the term “genotyping” refers to biochemical techniques and genome sequencing technologies for determining the nucleotide bases of a nucleic acid molecule (e.g., oligonucleotide, polynucleotide) molecule at one or more positions. Genotyping may involve genotyping array technologies, genome sequencing technologies, and/or statistical and computational data processing techniques. As used herein, the term “genetic data” refers to data produced by genotyping techniques and technologies, as described further herein.

As used herein, the terms “genotyping array”, “genotyping array technologies”, and “array genotyping” refer to microarrays targeting a number of mostly single nucleotide variants (e.g., SNP chips) and techniques for using the same. A genotyping array may determine a diploid genotype of an organism at a particular position on a chromosome. For example, a genotyping array could indicate with high confidence that an organism carries an A and a T allele at a position 100—one inherited from the father and one from the mother—on a chromosome and a G and a C at position 200. However, genotyping array technologies cannot determine whether the A and the G were inherited both from the same parent (e.g., the A at position 100 from the father and the G at position 200 from the father), or from different parents (e.g., the A at position 100 from the father and the G at position 200 from the mother). Thus, array genotyping alone cannot determine the haplotypes of an individual diploid organism.

As used herein, the terms “genome sequencing technologies”, “sequencing technologies”, “genome sequencing”, and “sequencing” refer to devices and biochemical techniques to automatically produce genetic sequence information from a sample containing nucleic acid (e.g., DNA, RNA, oligonucleotide, polynucleotide) molecules. Genome sequencing technologies typically read only short (e.g., 50 bp to 150 bp in length) segments of DNA, a process referred to as “short read sequencing”. Thus, like genotyping arrays, short read sequencing cannot determine the haplotypes of an individual organism. Even new sequencing approaches that produce significantly longer reads (e.g., Nanopore sequencing, HiFi-reads, or linked reads), referred to as “long read sequencing”, ameliorate, but under most circumstances, do not solve this problem.

As used herein, the terms “read”, “sequence read”, “sequencing read”, or “genetic read” refer to nucleotide sequences produced by any sequencing process described herein or known in the art. Reads can be generated from one end of nucleic acid fragments (single-end reads), and sometimes are generated from both ends of nucleic acids (paired-end reads, double-end reads). Reads can be obtained in a variety of ways, including but not limited to using sequencing techniques, using probes (e.g., in hybridization arrays or capture probes), and amplification techniques (e.g., polymerase chain reaction (PCR) or linear amplification using a single primer or isothermal amplification). Length of a sequence read is often associated with the particular sequencing technology used to produce the read. For example, short read sequencing techniques provide sequence reads that can vary in size from tens to hundreds of base pairs (bp). Illumina® parallel sequencing can provide sequence reads having lengths of about 200 bp or less. Nanopore sequencing, for example, can provide sequence reads that having lengths of tens to thousands of base pairs. A sequence read may refer to sequence information corresponding to a nucleic acid molecule or from a fragment of a nucleic acid molecule. Sequencing depth refers to the total number of usable reads from a sequencing technology or device.

As used herein, the term “coverage” refers to a relation between sequence reads and a reference genome. Coverage may be defined in various ways, including in terms of redundancy and in terms of percentage. Coverage defined in terms of redundancy refers to the number of reads that align to or cover a known reference genome. Coverage defined in terms of percentage refers to percent coverage of a known reference genome by reads. For example, if 90% of bases from a known reference genome are covered by reads, and 10% are not, then the coverage may be reported as 90% coverage.

As used herein, the terms “low-coverage sequencing” and “ultra-low-coverage sequencing” refer to genotyping processes that produce genetic data that does not comprehensively cover the genome. For example, ultra-low-coverage sequencing may refer to a set of genotype data in which each base is covered by less than one read on average.

As used herein, the terms “statistical data processing”, “statistical data analysis”, “computational data processing”, and “computational data analysis” refer to mathematical techniques and operations for organizing, converting, and/or analyzing data. For example, statistical data processing, as used herein, may include statistical phasing techniques that may be used in conjunction with short read sequencing, long read sequencing, or array genotyping techniques as described herein to estimate haplotypes of an individual organism. Such techniques may compare the genotype data to reference haplotypes to estimate the haplotypes present in an individual. However, these methods do not work perfectly over entire chromosomes and may result in errors, such as phasing errors. As used herein, the term “phasing error” refers to errors in which an estimated haplotype ends up being a mixture of both paternal and maternal haplotypes, instead of purely paternal or purely maternal haplotypes.

As used herein, the term “parental table” refers to a two-dimensional table of nucleotide sequences from a biological parent of an individual, with each of the two columns of the table giving the estimated parental haplotypes. Each individual may have two parental tables, one from each parent. As used herein, the term “inheritance vector” refers to lists indicating inheritance from each parent at a given position on the individual's chromosomes. For any given position, an inheritance vector denotes from which column of the two parental tables the individual's allele was copied. An inheritance vector contains only zeroes and ones, as described further herein.

As used herein, the terms “polygenic score” or “polygenic risk score” refer to functions of offspring genotype data that may be used to predict offspring traits (e.g. height) and disease risks (e.g. risk of Alzheimer's disease).

As used herein, the term “genetic disorder” refers to a disease, disorder, syndrome or condition arising from genetic alterations. Such genetic alterations may include, but are not limited to, chromosome microdeletions, chromosome aneuploidies, single gene conditions, or other genetic variations or mutations. A chromosome microdeletion is a deletion of a number of base pairs in chromosome which is too small (i.e., less than 5 million base pairs) to be detected by conventional imaging techniques. A chromosome aneuploidy is an occurrence of one or more extraneous or missing chromosomes. A single gene condition is a disease, disorder, syndrome or condition caused by genetic variation or mutation in a single gene. Genetic variation or mutation may refer to substitutions, insertions, deletions, or alterations of one or more nucleotides in a DNA or RNA sequence.

### Methods for Inferring Genotypes

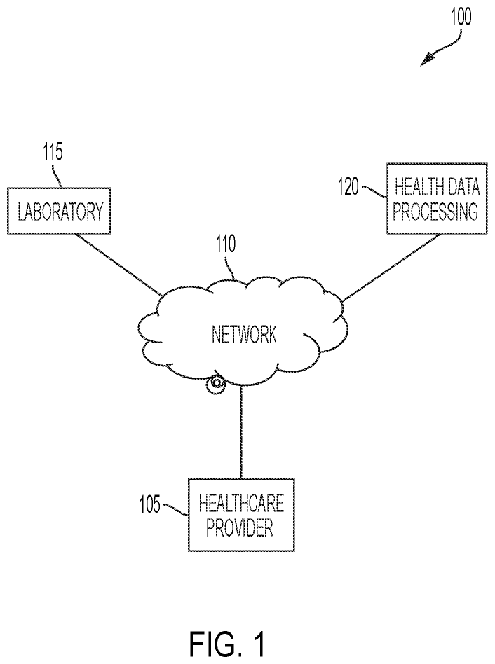

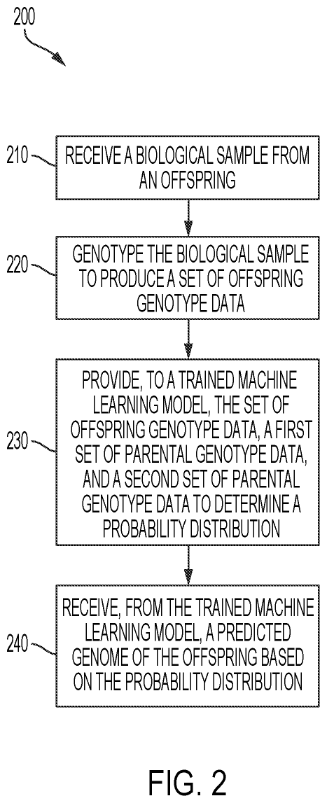

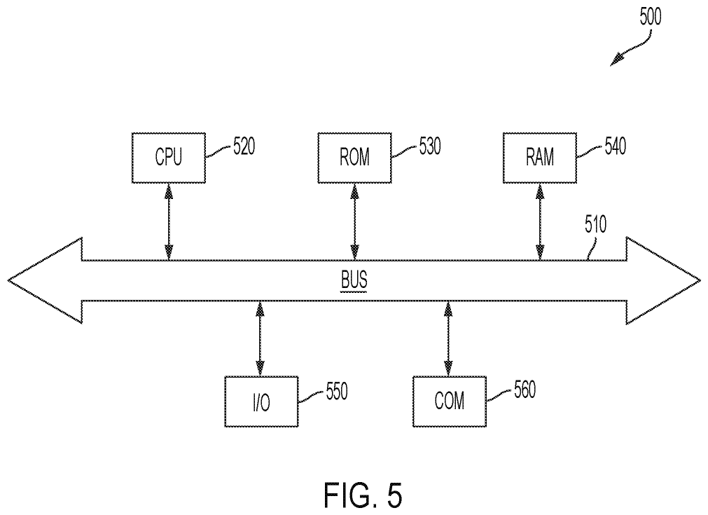

FIG. 1 is a block diagram of a system for inferring genotypes from a biological sample, according to techniques discussed herein. A person seeking genomic analysis of a biological sample of cells may engage a healthcare provider, for example, a doctor. The healthcare provider 105 may obtain or receive the biological sample of cells, and may analyze the biological sample(s) internally and/or engage a laboratory 115 to perform further analysis. The healthcare provider 105 may utilize devices to communicate across network 110 with devices at laboratory 115. The network communication devices may comprise computers, for example those discussed in FIG. 5. The network 110 may comprise the Internet, a Wi-Fi network, local area network (LAN), intranet, wireless, Bluetooth, Near Field Communication (NFC), and/or any wired or wireless data connection, or any combination thereof. While the laboratory, healthcare provider, and health data processors may be depicted as separate herein, they also may be the same entity. The healthcare provider 105 may provide data related to the biological sample(s), the process used to obtain the biological sample(s), information about the male or female parental contributors to the biological sample(s), demographic information, family history, medical history or information, etc., to the laboratory 115. The laboratory 115 may return test results pertaining to the biological sample(s), and may perform techniques discussed in relation to FIGS. 2, 3, and 4. Health data processing 120 may provide further data analysis, for example processing data from laboratory 115 to generate a probability distribution and/or a predicted genome of the offspring sample.

Described herein are methods for generating a predicted genome of an offspring based on genetic data produced from a biological sample from the offspring, as well as genetic data from both parents of the offspring.

FIG. 2 depicts an exemplary embodiment of the methods described herein. At step 210, a biological sample from an offspring may be received, for example at laboratory 115. A variety of techniques known in the art may be used to obtain the biological sample from the offspring. For example, a trophectoderm biopsy is a technique involving using a laser to create a small hole in the shell of an offspring pre-natal cellular cluster and removing a small number of cells from the trophectoderm component.

At step 220, the biological sample may be sequenced to produce a set of offspring sequence data. A variety of techniques known in the art may be used for sequencing, as described above, including genotype arrays, genome sequencing technologies, and statistical and computational data processing such as statistical phasing.

At step 230, the set of offspring sequence data, a first set of parental sequence data, and a second set of parental sequence data, may be provided to a machine learning model to determine a probability distribution, for example at health data processing 120. In some embodiments, the machine learning model may determine a probability distribution by: applying statistical and/or experimental phasing techniques to the first set of parental sequence data and the second set of parental sequence data to produce estimated first parental haplotypes and second parental haplotypes; determining which estimated parental haplotypes were inherited by the offspring based on offspring genotypes from offspring sequence data; inferring, at each genomic position, a probability that the offspring inherited each estimated parental haplotype based on estimating parental phasing quality and/or inferred phasing error rates; and determining a probability distribution based on the inferred probabilities at each genomic position.

Various probabilistic models and heuristic approaches known in the art may be used by the machine learning model, including, but not limited to, recurrent neural networks, heuristic approaches based on allele sharing with parental data, junction tree algorithms, Bayesian networks, and Hidden Markov Models.

### Hidden Markov Model (HMM)

In some embodiments, the HMM may comprise a set of “hidden states” that describe which estimated parental haplotypes the offspring inherits at each genomic position. For example, a particular position in the genome could be coded as (0,1), indicating the offspring inherits the first parent's (e.g., father's) estimated haplotype ‘0’ at the position and the second parent's (e.g., mother's) estimated haplotype ‘1’ at the same position. For each position, there are four possible inheritance patterns, and thus four possible hidden states, e.g., (0,0), (0,1), (1,0), and (1,1). Given the particular combination of estimated parental haplotypes inherited at a previous position, the HMM may describe the probability of an offspring having inherited a particular combination of parental haplotypes (coded by the four hidden states) at the next position.

The HMM includes an emission layer which describes the probability of observing genotype data in an offspring given a combination of estimated parental haplotypes that the offspring inherits. The emission layer can include probabilities that the offspring genotype data is erroneous, as determined from various types of error models. The choice of error model(s) differs depending on whether the offspring genotype was estimated via genotyping array or genomic sequencing technologies. The emission layer may further account for any missing offspring genotype data at any particular genomic position, while still including hidden states for that particular position.

An output of the HMM may be an inferred probability distribution over the particular combination of parental haplotypes that each offspring inherits given the genotype data of the offspring and the estimated parental haplotypes. The particular combination of estimated parental haplotypes that the offspring inherits can change between positions on a chromosome due to both recombination and phasing errors in the estimated parental haplotypes. The HMM can determine the probabilities of changes in estimated parental haplotype inheritance by the rate at which recombinations are known to happen between the positions during both male and female meiosis, according to reference to genetic maps that record the rate at which recombinations happen across the genome, and the rates of phasing errors in the estimated parental haplotypes.

The rate of phasing errors is generally unknown, and can be different for each parent, but may be inferred from the estimated parental haplotypes and the genotype data on one or more offspring. In some techniques, inferring a phasing error rate comprises finding a value of the maternal and paternal phasing error rates that maximizes the likelihood of the observed data, according to the HMM. This may, for example, be achieved by applying expectation-maximization type algorithms (e.g., Baum-Welch algorithm for HMMs) that find phasing error rates that maximize a combined likelihood of the observed data over the set offspring. In some techniques, inferring the phasing error rate comprises inferring the most likely inheritance vectors for each offspring given the phasing error rates, called Viterbi inheritance vectors. This may, for example, be achieved, by using dynamic programming algorithms such as the Viterbi algorithm. Given Viterbi inheritance vectors, one may find the phasing error rates that provide the same expected number of switches in inheritance vectors as observed in the Viterbi inheritance vectors. This process may be repeated until convergence of the phasing error rates. In some techniques, inferring the phasing error rate comprises using external data, heuristics, Bayesian priors, etc. on the phasing quality of the estimated parental haplotypes.

A probability distribution over the offspring inheritance vectors may be determined based on offspring genotype data, estimated parental haplotypes, and a genetic map and phasing error rates, as described herein. In some embodiments, the probability distribution over offspring inheritance vectors may be determined by application of a Forward-Backward-type algorithm, a recursive algorithm that determines both forward probabilities and backward probabilities at each genomic position. The forward probabilities at a position provide the joint probability of the observed offspring genotype data from the start of the chromosome to the particular genomic position, and the inheritance vector of the offspring at that genomic position. The forward probability at a genomic position can be computed according to the forward probabilities at the previous position and the probabilities of transitioning from one inheritance vector at a previous position to the given inheritance vector at the current genomic position, and the probability of observing the offspring genotype data given the inheritance vector at that position. The backward probabilities at a position give the probability of observing the genotype data on subsequent positions on the chromosome given the inheritance vector at the current position. The backward probabilities at a position can be computed from the backward probabilities at a subsequent position plus the probabilities of changing from the inheritance vector at the subsequent state to the current state, and probability of the observed genotype data on the offspring given the inheritance vector at the subsequent state.

To overcome numerical issues due to the multiplication of many small numbers, scaled forward and backward variables may be introduced to ensure that scaled forward variables sum to 1 at each genomic position. Another technique to overcome said numerical issues may be using logarithms to achieve numerical stability. Recursive computation of stabilized and/or scaled forward and backward variations allows for the computation of the probability of each inheritance vector at each genomic position for an offspring, given observed genotype data and phasing parameters.

In some other embodiments, dynamic programming type algorithms may be used, instead of or in addition to the Forward-Backward algorithm as described herein, to infer the most likely inheritance vector for each offspring given observed genotype data and phasing parameters. For example, a Viterbi algorithm may be used. However, such dynamic programming algorithms may not be preferred, as they may be unable to reflect uncertainty associated with which estimated parental haplotype is inherited by the offspring.

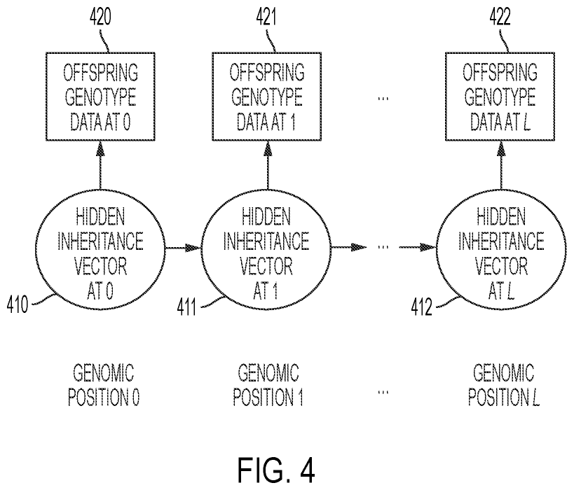

For the purposes of illustration only, FIG. 4 is a diagram depicting an exemplary HMM of the present disclosure. Observed offspring genotype data is modeled as emissions from the HMM. The selected positions may be those where at least one parent is heterozygous (i.e., has distinct alleles on the parent's paternal and maternal haplotypes) and at least one sequencing read from the offspring maps to that position. Alternatively, the selected positions may be those having a threshold imputation/genotyping quality and a threshold probability that at least one parent is heterozygous. For each genomic position (e.g., position 0, position 1, . . . , position L), the hidden states may be inheritance vectors 410, 411, 412. At each position, offspring genotype data 420, 421, 422 is emitted.

At step 240, a predicted genome and/or a probability distribution over may be received from the model, which may be an HMM and may be a machine learning model. In some embodiments, the predicted genome of the offspring received by the machine learning model may be generated by using the probability distribution over the offspring inheritance vectors combined with the estimated parental haplotypes to determine probability distributions over offspring genotype. For example, the genotypes may be coded numerically (e.g., 0, 1, 2, indicating 0, 1, or 2 copies of an allele at a position), which may be combined with probability that the offspring inherited 0,1, or 2 copies of an allele from its parents in order to computed an expected offspring genotype (dosage). Alternatively, Viterbi or other proposed inheritance vectors of the offspring can be used to give a specific inferred offspring genotype at a position.

In some embodiments, the predicted genome of the offspring received by the machine learning model may be generated by efficiently sampling offspring inheritance vectors given observed offspring genotype data, estimated parental haplotypes, and phasing error rates. The inheritance vectors may be sampled in proportion to their probability given the estimated parental haplotypes and the observed genotype data on the offspring. For example, a particular inheritance vector for an offspring at a terminal position on a chromosome may be sampled according to probabilities of the inheritance vector at that position. Probabilities of observed offspring genotype data, estimated parental haplotypes, and phasing error rates may be computed by multiplication of scaled forward and backward variables at that genomic position for each of the possible inheritance vectors at that position. An inheritance vector at a previous position can then be sampled according to the probability of the inheritance vector at the previous position given the inheritance vector sampled at the terminal position and the observed genotype data, estimated parental haplotypes, and phasing error rates. This probability can be computed efficiently using the scaled forward variables at the terminal position and previous position, the probability of the inheritance vector at the previous position given the terminal position, and the probability of the offspring genotype data given the sampled inheritance vector at the terminal position.

This process can be applied recursively to sample inheritance vectors backwards from the terminal position to starting position on the chromosome. A similar algorithm could proceed by sampling from the starting position of the chromosome. By applying such an algorithm repeatedly, one can sample a set of inheritance vectors that are sampled in proportion to their probability given the observed genotype data, estimated parental haplotypes, and phasing error rates. This enables the calculation of a probability distribution over arbitrary functions of the offspring inheritance vector, such as polygenic scores.

In some embodiments, the methods described herein may further comprise determining a probability over offspring polygenic scores, as described herein. In some embodiments, the method may comprise: using the probability distribution over estimated parental haplotypes to determine a most likely or expected genotype of each offspring (i.e., average dosage of an allele given the probability distribution over the estimated parental haplotypes) at each position in the genome for each offspring; and determining a probability over offspring polygenic scores based on the most likely or expected genotype of each at each position in the genome for each offspring. In some embodiments, the method may comprise: sampling inheritance vectors in proportion to their probability given the estimated parental haplotypes and the observed genotype data on the offspring; and determining a probability over offspring polygenic scores based on the sampled inheritance vectors. In some embodiments, the method may comprise: using the probability distribution over estimated parental haplotypes to determine a most likely or expected genotype of each offspring at each position in the genome for each offspring; sampling inheritance vectors in proportion to their probability given the estimated parental haplotypes and the observed genotype data on the offspring; and determining a probability over offspring polygenic scores based on the most likely or expected genotype of each at each position in the genome for each offspring and the sampled inheritance vectors

In some embodiments, the methods described herein may further comprise determining a prediction of an offspring's traits and/or disease risks based on the estimated parental haplotypes, observed genotype data on the offspring, information on the diseases and phenotypes of the parents and other relatives of the offspring, sampled inheritance vectors, and/or a probability over offspring polygenic scores, as described herein.

In some embodiments, the method described herein may further comprise selecting an offspring according to an arbitrary utility function based on a prediction of an offspring's traits and/or disease risks, as described herein. In some aspects, selecting offspring may comprise selecting a group of preimplantation stage cell clusters cultivated as part of an assisted reproduction technique.

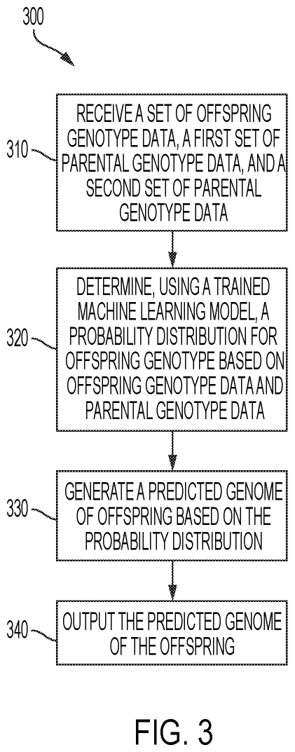

FIG. 3 depicts another exemplary method 300 of the present disclosure. At step 310, a set of offspring genotype data, a first set of parental genotype data, and a second set of parental genotype data may be received. At step 320, a probability distribution for offspring genotype data may be determined, using a machine learning model, based on offspring genotype data and parental genotype data, as described herein. In some embodiments, the machine learning model is an HMM, as described herein and as illustrated in FIG. 4, as may be used by health data processor 120. At step 330, a predicted genome of the offspring may be generated, using the machine learning model, based on the probability distribution, as described herein. At step 340, the predicted genome of the offspring may be output. In some embodiments, the output may comprise transmitting the predicted genome, for example to a patient or healthcare provider 105.

For the purposes of illustration, an overview of an exemplary HMM algorithm is now described. Meiosis events are independent between parents and between offspring, implying that offspring haplotypes are conditionally independent given the true parental haplotypes. However, when haplotypes are estimated with phasing errors, the offspring haplotypes are not conditionally independent given the estimated parental haplotypes. This is because a phasing error in a parental haplotype causes all the offspring inheritance vectors from that parent to switch which estimated haplotype in the parent they are copying from (barring an unlikely coincidence with a true recombination).

The present embodiment may utilize an approach that assumes the phasing of the estimated parental haplotypes is true. The offspring may then be treated as independent, giving a complexity that is linear in the number of offspring. However, to accommodate the fact that there are likely to be phasing errors in the parental haplotypes, the present embodiment may utilize a Baum-Welch algorithm to obtain maximum likelihood estimates of the paternal and maternal phasing rates. Given these parameters, the HMM may obtain the maximum likelihood inheritance vectors by a Viterbi algorithm and obtains posterior distributions over inheritance vectors by a Forward-Backward algorithm. Given outputs of the algorithm (Viterbi inheritance vectors, posterior probability distributions), phasing errors in the estimated parental haplotypes may be corrected through various approaches. After improving phasing in the estimated parental haplotypes, the Baum-Welch may be rerun to infer the new phasing-error rate (which will be lower if the phasing has been improved but will likely remain non-zero) before performing inference on the offspring inheritance vectors.

In an implementation, the present embodiment may involve the following steps:
using a Baum-Welch algorithm, treating each offspring independently, to obtain maximum likelihood estimates of the paternal and maternal phasing error rates, λₚ and λₘ;
generating, using a Forward-Backward algorithm, a probabilistic inference on offspring inheritance vectors; and/or inferring, using a Viterbi algorithm, the most likely offspring inheritance vectors;
correcting for phasing errors in the estimated parental haplotypes, using the probabilistic inference on the offspring inheritance vectors and/or the most likely offspring inheritance vectors;
determining, using the Baum-Welch algorithm treating each offspring independently, corrected paternal and maternal phasing error rates based on the corrected estimated parental haplotypes;
performing, using the Forward-Backward algorithm, posterior inference on offspring inheritance vectors based on the corrected paternal and maternal phasing errors; and
performing, using the Forward-Backward algorithm, posterior decoding and/or sampling of inheritance vectors from the posterior distribution to obtain posterior means, variances, and samples of functions of offspring inheritance vectors, such as polygenic scores.

Another exemplary implementation can be found in U.S. Patent Provisional Application No. 63/674,912, the entirety of which is incorporated by reference herein.

Exemplary Forward-Backward algorithms, Baum-Welch algorithms, and Viterbi algorithms are described in, e.g., DURBIN et al., “Biological sequence analysis: probabilistic models of proteins and nucleic acids”, Cambridge University Press, 1998, the entirety of which is incorporated by reference herein.

### Techniques for Correction of Phasing Rates

Various techniques may be used for the correction of phasing errors in parental haplotypes as described herein, including but not limited to heuristic techniques, dynamic programming type algorithms, and full probabilistic models.

In some embodiments, phasing errors may be corrected using heuristic techniques that take advantage of the fact that phasing errors in estimated parental haplotypes will lead to shared switches in inferred/estimated offspring inheritance vectors. Recombinations happen independently between offspring and the probability of two offspring's inheritance vectors switching at a similar position in the genome due to recombination is typically low. For example, the (scaled or unscaled) forward and backward variables may be used to compute the probability of a change in inheritance vector between two positions in the genome. If multiple offspring exhibit substantial total probability of changing inheritance vectors between two nearby positions in the genome, this may indicate a phasing error in one or both parents. By setting a threshold for the sum of the probability of a change in inheritance vectors across multiple offspring, one may estimate the position of the phasing error in a parent and then correct the parental haplotype to remove the phasing error or reduce its size and therefore impact. Other statistics may be used, such as statistics derived from the scaled or unscaled forward backward variables and those derived from the Viterbi inheritance vectors. Visual inspection of offspring Viterbi inheritance vectors and/or probabilities over inheritance vectors may be used to identify likely phasing errors in parental haplotypes that may then be manually corrected. Other information, such as information on the quality and/or confidence or the statistical and/or experimental phasing of the estimated parental haplotypes, may also be incorporated into heuristic decision rules.

In some embodiments, phasing errors in parental haplotypes may be corrected using dynamic programming type algorithms that find the estimated parental haplotypes that minimize the number of switches in the offspring inheritance vectors. This could be the expected number of switches according to the HMM probabilities or the number of switches according to the Viterbi inheritance vectors. Alternatively, the dynamic programming algorithms could be used to maximize a likelihood function that penalizes excessive switching in inheritance vectors above and beyond those expected due to recombinations.

In some embodiments, phasing errors in parental haplotypes may be corrected using full probabilistic models that perform joint inference over parental haplotypes and offspring inheritance vectors. Parental phasing errors may be corrected by finding the parental haplotypes that maximize the probability of the observed data given the parental haplotypes.

In some embodiments, phasing errors in parental haplotypes may be corrected using heuristic techniques that leverage the stark disparity between switch errors and true recombinations. If high-quality genotyping array or sequencing data on a single offspring is available, it may be used to identify and fix all parental phasing errors by assuming that every single switch in the offspring inheritance vector is due to phasing errors in parental haplotypes rather than recombination. This will induce phasing errors at all positions where actual recombinations happened and therefore lower the phasing error rate on average to the recombination rate.

### Exemplary Embodiment

Provided herein is an exemplary embodiment, which is not intended to be limiting in any way. Raw genetic sequence data (reads) from an individual corresponding to sequences at positions on each of a number of chromosomes that offer information about which parental genetic sequences were inherited by the individual. Genetic sequences at these positions are represented by the four bases (nucleotides): adenine (A), thymine (T), guanine (G), and cytosine (C). These positions are determined by identifying positions which satisfy two conditions: the presence of a sequence read at that position, and heterogeneity in the bases of the parental genetic sequences for one or both parents, referred to as “heterozygosity”. Heterozygosity in a parent at a position indicates that the parent carries different nucleotide bases on each of their haplotypes at that position, e.g., an ‘A’ and a ‘T’ from each haplotype. Within the model, these sequences may be recorded in two separate two-dimensional tables, one table for chromosomes of a first parent (e.g., mother) and one table for chromosomes of a second parent (e.g., father). For example, such a table may comprise [A,T], [A,A], [T,G], [C,T].

In some embodiments, the offspring genetic sequence data may be derived from outputs from a Hidden Markov Model (HMM), as described herein. Hidden states in the HMM may be determined by two lists, one for each parent. Each list may contain only zeros and ones, denoting from which column of the respective parent's table the offspring copies, and thereby indicating from the estimated parental haplotype the offspring inherits from each parent at any given genomic position. The HMM may assume that a hidden state of the model at a given position (denoted by the inheritance vectors) depends only on a hidden state at a previous position. In this manner, the HMM may determine a probability distribution of possible hidden states (or inheritance vectors) based on the observed parent and offspring genotype data, which in turn provides a probability distribution over nucleotide bases inherited from each parent.

In some embodiments, the occurrence of an offspring sequence read deriving from a parent's genetic sequence may be modeled probabilistically. For example, it may be assumed that a sequence read from the offspring has an equal likelihood of originating from either parent (e.g., mother or father). Additionally, there is a possibility that the sequence read from the offspring is an error, with the three possible incorrect nucleotide bases being assigned a probability if the read is erroneous. Sequencing errors can be considered as independent across reads. The probability of observing a set of sequence reads from an offspring, given the inheritance vectors, may be calculated by multiplying the probabilities for all reads together. Reads from different positions may be assumed to be independent, given the inheritance vectors and estimated parental haplotypes.

In some embodiments, the probability of changing from one state to another at a genomic position in the HMM may then be estimated based on a probability of recombination between the positions on the chromosome, which can be inferred from reference genetic maps, including those that are publicly available. Inheritance from both parents, e.g. mother and father, may be modeled separately using the female and male genetic maps, respectively. If available, ancestry-specific maps may be used. Phasing errors, which may affect the inheritance vectors, may be modeled based on estimates from genetic map positions.

In some embodiments, as discussed above, the probability of different inheritance vectors, based on parent and offspring genotype data, may be calculated using a Forward-Backward algorithm. This technique allows discovery of the most likely sequence of states given observed data. For example, the Forward-Backward algorithm may perform two passes: a forward pass and a backward pass, calculating a probability of observing the reads up to a certain point and ending in a specific state for the forward pass, and from a given state to the end of the sequence for the backward pass. Upon completion of the forward and backward passes, the Forward-Backward algorithm may calculate a posterior probability of each state at each position by multiplying the corresponding forward and backward probabilities and normalizing by the total probability of the observed data. The posterior probabilities may then be used to calculate the probability of the inherited alleles from each parent at each position. For positions not included in the initial set used in the HMM, the posterior probability may be estimated using interpolation (e.g., linear interpolation) based on the nearest positions that were included. Since the calculation may involve multiplying numerous probabilities, this could lead to numerical underflow. To prevent this issue, the probabilities may be scaled at each iteration by a factor that ensures the sum of the forward probabilities at each position equals 1, with the backward probabilities scaled using the same factors.

### Computer-Implemented Systems and Devices for Inferring Genotypes

FIG. 5 illustrates an example computer-implemented system or device 400 that may execute techniques presented herein, and may correspond to devices shown in FIG. 1, such as devices associated with laboratory 115, health care provider 105, health data processing 120, and/or network 110. Device 500 may include a central processing unit (CPU) 520. CPU 520 may be any type of processor device including, for example, any type of special purpose or a general-purpose microprocessor device. As will be appreciated by persons skilled in the relevant art, CPU 520 also may be a single processor in a multi-core/multiprocessor system, such system operating alone, or in a cluster of computing devices operating in a cluster or server farm. CPU 520 may be connected to a data communication infrastructure 510, for example a bus, message queue, network, or multi-core message-passing scheme.

Device 500 may also include a main memory 540, for example, random access memory (RAM), and also may include a secondary memory 530. Secondary memory 530, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 530 may include similar means for allowing computer programs or other instructions to be loaded into device 500. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 500.

Device 500 also may include a communications interface (COM) 560. Communications interface 560 allows software and data to be transferred between device 500 and external devices. Communications interface 560 may include a modem, a network interface (such as an Ethernet card), a communications port, a PCMCIA slot and card, or the like. Software and data transferred via communications interface 560 may be in the form of signals, which may be electronic, electromagnetic, optical or other signals capable of being received by communications interface 560. These signals may be provided to communications interface 560 via a communications path of device 500, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 500 may also include input and output ports 550 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

### Additional Applications

The methods, systems, and devices described herein may be used for any application in which offspring and parental genetic data is available and in which the offspring genetic data is incomplete, and even in circumstances where very little genetic data is available from the offspring.

In some embodiments, the methods, systems, and devices described herein can greatly enhance and open new fields of assisted reproduction technologies. However, they are not limited to use with human reproduction. For example, the methods and systems described herein may be used in animal breeding programs, where enhancement of genetic selection procedures can lead to improved outcomes.

In some embodiments, the methods described herein may further comprise determining a probability that the subject has or will develop one or more genetic disorders. In some embodiments, the one or more genetic disorders arise from chromosome microdeletions, chromosome aneuploidies, single gene conditions, or other genetic variations. In some embodiments, the one or more genetic disorders includes: Angelman Syndrome, DiGeoge/VCF, Prader-Willi Syndrome, Williams Syndrome, Down Syndrome, Klinefelter Syndrome, Trisomy 18, Trisomy 13, Turner Syndrome, Ehlers-Danlos Syndrome, Fragile X Syndrome, Marfan Syndrome, Neurofibromatosis Type 1, Noonan Syndrome, Osteogenesis Imperfecta, Phenylketonuria, Rett Syndrome, Smith-Lemli-Opitz Syndrome, Tuberous Sclerosis, and Russell-Silver Syndrome.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

1. A method for generating a predicted genome of a subject, the method comprising:
receiving a biological sample from the subject, wherein the subject is an offspring of a first parent and a second parent;
genotyping the biological sample to produce offspring genotype data;
providing, to a machine learning model, the offspring genotype data, a first parental genotype data from the first parent, and a second parental genotype data from the second parent to determine a probability distribution; and
receiving, from the machine learning model, a predicted genome of the subject based on the probability distribution.

2. The method of claim 1, wherein the first parental genotype data comprises a complete genome of the first parent, the second parental genotype data comprises a complete genome of the second parent, and the offspring genotype data comprises a partial genome of the subject.

3. The method of claim 1, wherein the machine learning model comprises a Hidden Markov Model.

4. The method of claim 1, wherein the offspring genotype data comprises an average coverage of less than one read per base position.

5. The method of claim 1, wherein the parental genotype data comprises information at one or more additional base positions than the offspring genotype data.

6. The method of claim 1, further comprising:
determining a probability that the subject has or will develop one or more genetic disorders.

7. The method of claim 6, wherein the one or more genetic disorders arise from chromosome microdeletions, chromosome aneuploidies, single gene conditions, or other genetic variations.

8. The method of claim 6, wherein the one or more genetic disorders includes: Angelman Syndrome, DiGeoge/VCF, Prader-Willi Syndrome, Williams Syndrome, Down Syndrome, Klinefelter Syndrome, Trisomy 18, Trisomy 13, Turner Syndrome, Ehlers-Danlos Syndrome, Fragile X Syndrome, Marfan Syndrome, Neurofibromatosis Type 1, Noonan Syndrome, Osteogenesis Imperfecta, Phenylketonuria, Rett Syndrome, Smith-Lemli-Opitz Syndrome, Tuberous Sclerosis, and Russell-Silver Syndrome.

9. The method of claim 1, further comprising:
receiving, from the machine learning model, at least one predicted polygenic risk score for the subject based on an expected genotype at each base position in the predicted genome and/or based on sampled inheritance vectors.

10. The method of claim 9, wherein the at least one predicted polygenic risk score for the subject is determined by:
using the probability distribution to determine an expected genotype of each offspring at each position in a genome for one or more offspring of the first parent and the second parent, and/or sampling inheritance vectors in proportion to their probability based on estimated parental haplotypes and observed genotype data from the one or more offspring;
generating one or more offspring polygenic risk scores based on the expected genotype at each position in a genome for one or more offspring and/or based on the sampled inheritance vectors; and
determining the at least one predicted polygenic risk score for the subject based on the one or more offspring polygenic risk scores.

11. A method for generating a predicted genome of a subject, the method comprising:
receiving an offspring genotype data of the subject, a first parental genotype data of a first parent, and a second parental genotype data, wherein the subject is an offspring of the first parent and the second parent;
determining, using a machine learning model, a probability distribution for an offspring genotype based on the offspring genotype data, the first parental genotype data, and the second parental genotype data;
generating, using the machine learning model, a predicted offspring genome based on the probability distribution; and
outputting the predicted offspring genome.

12. The method of claim 11, wherein the first parental genotype data comprises a complete genome of the first parent, the second parental genotype data comprises a complete genome of the second parent, and the offspring genotype data comprises a partial genome of the subject.

13. The method of claim 11, wherein the offspring genotype data is produced by array genotyping and/or sequencing of a biological sample from the subject.

14. The method of claim 11, wherein the machine learning model comprises a Hidden Markov Model.

15. The method of claim 11, wherein the offspring genotype data comprises an average coverage of less than one read per base position.

16. The method of claim 11, wherein the parental genotype data comprises information at one or more additional base positions than the offspring genotype data.

17. The method of claim 11, further comprising:
analyzing the predicted offspring genome to determine a probability that the subject has or will develop one or more genetic disorders.

18. The method of claim 17, wherein the one or more genetic disorders arise from chromosome microdeletions, chromosome aneuploidies, single gene conditions, or other genetic variations.

19. The method of claim 17, wherein the one or more genetic disorders includes: Angelman Syndrome, DiGeoge/VCF, Prader-Willi Syndrome, Williams Syndrome, Down Syndrome, Klinefelter Syndrome, Trisomy 18, Trisomy 13, Turner Syndrome, Ehlers-Danlos Syndrome, Fragile X Syndrome, Marfan Syndrome, Neurofibromatosis Type 1, Noonan Syndrome, Osteogenesis Imperfecta, Phenylketonuria, Rett Syndrome, Smith-Lemli-Opitz Syndrome, Tuberous Sclerosis, and Russell-Silver Syndrome.

20. The method of claim 11, further comprising:
outputting at least one predicted polygenic risk score for the subject, wherein outputting the at least one predicted polygenic risk score of the subject comprises:
using the probability distribution to determine an expected genotype of each offspring at each position in a genome for one or more offspring of the first parent and the second parent, and/or sampling inheritance vectors in proportion to their probability based on estimated parental haplotypes and observed genotype data from the one or more offspring;
generating one or more offspring polygenic risk scores based on the expected genotype at each position in a genome for one or more offspring and/or based on the sampled inheritance vectors;
determining the at least one predicted polygenic risk score for the subject based on the one or more offspring polygenic risk scores; and
outputting the at least one predicted polygenic risk score.
